(19) United States
(12) Patent Application Publication
(10) Pub. No.: US 2010/0003681 A1
(43) Pub. Date: Jan. 7, 2010

(54) GENE POLYMORPHISM USEFUL FOR ASSISTANCE/THERAPY FOR SMOKING CESSATION

(76) Inventors: Junichi Azuma, Osaka (JP); Yasushi Fujio, Osaka (JP); Chieko Taniguchi, Osaka (JP); Tsuyoshi Fukuda, Osaka (JP); Makiko Maeda, Osaka (JP); Tomoko Kubota, Osaka (JP); Etsuko Tange, Osaka (JP); Risa Ueki, Osaka (JP)

Correspondence Address: WENDEROTH, LIND & PONACK, L.L.P., 1030 15th Street, N.W.,, Suite 400 East, Washington, DC, 20005-1503, US

(21) Appl. No.: 12/310,114

(22) PCT Filed: Aug. 9, 2007
(86) PCT No.: PCT/JP2007/065605

§ 371 Date: Feb. 11, 2009

(30) Foreign Application Priority Data
Aug. 11, 2006 (JP) 2006-219930

(51) Int. Cl. C12Q 1/68

(52) U.S. Cl. 435/6

(57) ABSTRACT

Disclosed is a method for determination of a smoking trend in a subject which is useful for the assistance or therapy for smoking cessation, which comprises the steps of detecting one or more polymorphisms occurring on one or more genes selected from the genes (1) to (3) or a genome or in the neighborhood of the genes or the genome and determining the smoking trend based on the results of the detection: (1) nicotinic acetylcholine receptor β2 subunit (CHRNB2) gene; (2) serotonin transporter (SLC6A4(5HTT)) gene; and (3) nicotinic acetylcholine receptor α4 subunit (CHRNA4) gene.

### TECHNICAL FIELD

The present invention relates to a method for determining the smoking tendencies of a test subject (such as their level of nicotine dependence, severity of nicotine withdrawal symptoms during smoking cessation, difficulty of sustainable smoking cessation, etc.) based on the gene polymorphism of the test subject. Particularly, the present invention provides a technique useful for general smoking cessation guidance, such as smoking cessation assistance/therapy etc.

### BACKGROUND ART

According to an estimate by the World Health Organization, smoking-related deaths in Japan increased to 114,200 in the year of 2000, and this upward tendency is likely to continue. Given these circumstances, the Ministry of Health, Labor and Welfare and medicine-related organizations have been strongly promoting smoking cessation as a policy to prevent smoking-related diseases, taking special notice of improvement in the smoking cessation rate. However, many smokers are dependent on nicotine both mentally and physically, and it is difficult for them to accomplish smoking cessation.

Nicotine dependence caused by smoking is already recognized as an illness in both Europe and America. The DSM-IV (Diagnostic and Statistical Manual of Mental Disorders) provided by the American Psychiatric Association categorizes the illness as “nicotine dependence” among nicotine use related diseases. In light of such global movements, a “smoking cessation guideline” was created in 2005 as the first Japanese smoking therapy instruction by a joint research group of nine societies, including the Japanese Circulation Society. Further, in 2006, an announcement that smoking cessation assistance that is administered by doctors will be covered by public health insurances was made. These movements were aimed at developing and expanding smoking cessation programs based on behavioral science and pharmacology in view of chemical dependency, improving the quality of health guidance and smoking cessation guidance in the health/medical fields, and increasing the rate of successful smoking cessations. The development of smoking cessation assistance based on scientific evidence will be an important contribution to the promotion of these smoking cessation policies.

In the meantime, the inventors of the present invention reported that the individual differences of nicotine kinetics, which derive from differences in gene polymorphism of the drug metabolizing enzyme CYP2A6, have some influence on smoking habits, levels of nicotine dependence, and the severity of nicotine withdrawal symptoms during smoking cessation (Non-patent Document 1). This finding suggested that the data regarding CYP2A6 gene polymorphism can be a good factor for smokers to understand their smoking habits or levels of nicotine dependence, and can also be useful in the prediction of the severity of nicotine withdrawal symptom during smoking cessation.
Non-patent Document 1: Pharmacogenomics J. March-April 2006; 6(2):115-9

### DISCLOSURE OF THE INVENTION

### Technical Problem

An object of the present invention is to search for a genetic factor involved in the individual differences of diathesis in smoking habits and smoking cessation, so as to realize smoking cessation assistance that is effective and is based on scientific evidence, and to establish and provide, based on the obtained result, a method for determining tendencies in the smoking behavior of a test subject useful for smoking cessation assistance and therapy, based on gene polymorphism information.

As described, the information regarding the polymorphism of the drug metabolizing enzyme CYP2A6 is useful for smoking cessation assistance and therapy; however, a group showing high levels of CYP2A6 activity, which is greatly involved in nicotine dependence, still showed variations in nicotine dependence among individuals. This indicates that the estimation of nicotine dependence based solely on drug kinetics is not sufficiently reliable. In view of this problem, an object of the present invention is to estimate and search for gene polymorphisms involved in nicotine dependence or nicotine withdrawal symptoms based on pharmacodynamic studies, and to provide a process for determining the smoking tendencies of a test subject that is useful for smoking cessation assistance and therapy based on the polymorphism confirmed to be involved in nicotine dependence or nicotine withdrawal symptoms.

### Technical Solution

When a person smokes a cigarette, nicotine reaches the cerebral blood vessel barrier within several seconds, activating the nicotine acetylcholine receptor (nAChR), thus releasing various neurotransmitters and causing neural activation.

The reward system of the dopamine neuron (A10) in the brain and other various neurons, such as glutamine neurons and GABA neurons, which adjust the dopamine neuron (A10), play important roles in the formation of nicotine dependence.

Based on this, the inventors of the present invention focused on the dopamine A10 neuron, which plays an important role in the formation of nicotine dependence, and selected 37 gene polymorphisms in 18 genes that are considered to have effects on its neurologic functions. The inventors examined the correlation between the polymorphisms of these genes and the individual differences in smoking behavior.

As detailed in the later-described Example, the inventors found that several gene polymorphisms, including a gene polymorphism (10160 A/C polymorphism) in the 5th intron of the nicotine acetylcholine receptor β2 subunit (CHRNB2) gene, are involved in the individual differences in smoking behavior and that these gene polymorphisms are useful for determining a tendency in the smoking behavior of a test subject such as the level of nicotine dependence, severity of nicotine withdrawal symptoms during smoking cessation, difficulty in sustaining smoking cessation etc. The inventors also found that the result of this determination can be used as important indices for individualized cessation assistance and therapy. The present inventions have been completed accordingly.

Specifically, the present invention includes the inventions as shown in the following items A) to J), which are industrially applicable.
A) A process of determining a tendency in the smoking behavior of a test subject, the process comprising examining a single or plural polymorphisms in one or plural genes selected from the genes (1) to (3) or in the vicinity of the genes in the genome; and, based on the examination result, determining a tendency in the smoking behavior of a test subject useful for smoking cessation assistances or smoking cessation therapies,

(1) nicotine acetylcholine receptor β2 subunit (CHRNB2) gene

(2) serotonin transporter (SLC6A4(5HTT)) gene

(3) nicotine acetylcholine receptor α4 subunit (CHRNA4) gene
B). The process according to item A), wherein the tendency in the smoking behavior of a test subject is at least one member selected from the group consisting of nicotine dependence level, severity of nicotine withdrawal symptoms, and difficulty in sustaining smoking cessation.
C). The process according to Items A) or B), comprising the step of directly or indirectly examining rs12072348 polymorphism (10160 A/C polymorphism) in the fifth intron of the CHRNB2 gene.
D). The process according to Items A) or B), comprising the step of directly or indirectly examining at least one polymorphism selected from the group consisting of an LPR polymorphism in a promoter region, a VNTR polymorphism in the second intron, and a the rs717742 polymorphism in the eighth intron of the SLC6A4 (5HTT) gene.
E). The process according to Items A) or B), comprising the step of directly or indirectly examining at least one polymorphism selected from the group consisting of an rs1044397 polymorphism in the fifth exon, and an rs2273504 polymorphism in the second intron of the CHRNA4 gene.
F). A smoking cessation assistance program for causing a computer to execute a determination of a tendency in the smoking behavior of a test subject, according to one of the processes of Items A) to E).
G). The process according to any one of Items A) to E), wherein the examination of polymorphism comprises the step of amplifying a gene region including the polymorphism site using a genomic DNA prepared from the test subject as a template; and identifying a genotype based on the amplified fragment.
H). The process according to any one of Items A) to E), wherein the examination of polymorphism is performed with a gene polymorphism examination tool, such as a DNA chip.
I). A gene polymorphism examination oligonucleotide to be used in the process of Item H).
J). A gene polymorphism examination kit to be used in any one of the processes of Items A) to E).

### EFFECT OF INVENTION

The present invention allows for the determination of a tendency in the smoking behavior of a test subject, such as the level of nicotine dependence, severity of nicotine withdrawal symptoms or difficulty in sustaining smoking cessation, based on the gene polymorphism, thereby providing important indices for conducting individually suitable smoking cessation assistance/therapy.

More specifically, understanding of tendencies in smoking behavior is important not only for smokers who are trying to accomplish smoking cessation, but also for those who assist them.

Understanding of the tendencies of smoking behavior is also useful to plan the details of courses/procedures of the smoking cessation assistance/therapy, for example, adjustment of the size, number and application time of a smoking cessation patch, thereby contributing to the planning of more individualized smoking cessation assistance.

### BRIEF DESCRIPTION OF THE DRAWINGS

FIG. 1 is a graph according to an Example of the present invention, showing a correlation between nicotine dependence and smoker's age.

FIG. 2 is a graph according to an Example of the present invention, showing a correlation between an LPR/VNTR polymorphism of serotonin transporter (5HTT) and nicotine dependence.

FIG. 3 is a graph according to an Example of the present invention, showing a correlation between an A10160C polymorphism of a nicotine acetylcholine receptor β2 subunit (CHRNB2) and nicotine dependence.

FIG. 4 is a graph according to an Example of the present invention, showing a correlation between an A10160C polymorphism of a nicotine acetylcholine receptor β2 subunit (CHRNB2) and the severity of nicotine withdrawal symptoms (withdrawal score 1) during smoking cessation.

FIG. 5 is a graph according to an Example of the present invention, showing a correlation between an A10160C polymorphism of a nicotine acetylcholine receptor β2 subunit (CHRNB2) and the severity of nicotine withdrawal symptoms (withdrawal score 2) during smoking cessation.

FIG. 6 is a graph according to an Example of the present invention, showing a correlation between an A10160C polymorphism of a nicotine acetylcholine receptor β2 subunit (CHRNB2) and smoking cessation sustainability.

### BEST MODE FOR CARRYING OUT THE INVENTION

A preferred embodiment of the present invention is described below. In the specification and drawings, bases, amino acids, etc. may be written as brevity codes according to the IUPAC-IUB Commission on Biochemical Nomenclature or using known other brevity codes in the field.

In the following, numbers, values or other information regarding gene polymorphisms and gene sequences etc. will be understood in reference to the numbers, values, etc. in the main gene databases available on the web page of the US National Center for Biotechnology Information (NCBI) or the like.

### [1] Determination Method of the Present Invention

As described, the determination method of the present invention comprises examining single or plural polymorphisms in single or plural genes selected from the following group of genes (1) to (3) or those existing in the vicinity of these genes in the genome; and based on the result, and determining a tendency in the smoking behavior of a test subject useful for smoking cessation assistance or smoking cessation therapy.
(1) Nicotine acetylcholine receptor β2 subunit (CHRNB2) gene
(2) Serotonin transporter (SLC6A4(5HTT)) gene
(3) Nicotine acetylcholine receptor α4 subunit (CHRNA4) gene

The following specifically explains a process of determining a tendency the smoking behavior of a test subject by examining the respective polymorphisms of the genes (1) to (3).

### (1) Nicotine Acetylcholine Receptor β2 Subunit (CHRNB2) Gene

For example, as detailed below, the tendency in the smoking behavior of a test subject can be determined by directly or indirectly examining the rs12072348 polymorphism (10160A/C polymorphism) that exists in the fifth intron of the CHRNB2 gene.

More specifically, as detailed in the later-described Example, when the test subject (Genotype C/C or A/C: a C allele carrier) had C alleles, in which the base on the polymorphism site is “C”, the following tendencies for smoking behavior were indicated.
(i) Nicotine dependence: weak
(ii) Nicotine withdrawal symptoms during smoking cessation: weak
(iii) Sustainable smoking cessation: difficult

In contrast, when the test subject (genotype A/A) had homozygous A-alleles, in which the base on the polymorphism site is “A”, the following tendencies for smoking behavior were obtained.
(i) Nicotine dependence: strong
(ii) Nicotine withdrawal symptoms during smoking cessation: strong
(iii) Sustainable smoking cessation: easy

Based on these examination results, the tendency in the smoking behavior of the test subject can be determined according to whether the genotype of the rs12072348 polymorphism is A/A, or a C allele carrier (C/C or A/C).

### (2) Serotonin Transporter (SLC6A4(5HTT)) Gene

For example, as detailed below, the tendency in the smoking behavior of a test subject can be determined by directly or indirectly examining the LPR polymorphism that exists in the promoter region of the SLC6A4 (5HTT) gene, and the VNTR polymorphism that exists in the second intron.

The LPR polymorphism has three kinds of alleles called S (short), L (long), and XL (extra long) (J Neurochem 1996; 66:2621-4., Biol Psychiatry 1998; 44:179-92). The VNTR polymorphism has two kinds of alleles called “s” (short) and “l” (long) (Am J Med Genet 2002; 114:323-8, Lancet 1996; 347:731-3).

As detailed in the later-described Example, the following test result regarding the tendency in the smoking behavior was obtained for a test subject who had an L allele carrier LPR polymorphism (L/S, L/L, or XL/) and an l/s type VNTR polymorphism.

(i) Nicotine Dependence: Strong

Contrastively, the remaining test subjects all had weak nicotine dependences.

Based on these examination results, the tendency in the smoking behavior of the test subject can be determined according to whether the test subject has both an L allele carrier LPR polymorphism (L/S, L/L, or XL/L) and a l/s type VNTR polymorphism or not.

Furthermore, as detailed below, the tendency in the smoking behavior of a test subject can be determined by directly or indirectly examining the rs717742 polymorphism that exists in the eighth intron of the SLC6A4 5HTT) gene.

More specifically, when the test subject (genotype T/T or A/T: a T allele carrier) had T alleles, in which the base on the polymorphism site is “T”, the following test result was obtained.

(i) Nicotine dependence: strong In contrast, when the test subject (genotype A/A) had homozygous A alleles in which the base on the polymorphism site is “A”, the following test result was obtained.

(i) Nicotine dependence: weak

Based on these examination results, the tendency in the smoking behavior of the test subject can be determined according to whether the genotype of the rs717742 polymorphism is A/A, or a T allele carrier (T/T or A/T).

Furthermore, among the test subjects having the genotype A/A the rs717742 polymorphisms, the number of those who failed a two-month smoking cessation was significantly higher than the number of those who accomplished it (Table 1 below); this shows that sustainable smoking cessation was “difficult” for the A/A type test subjects. As such, using the examination results, the smoking cessation sustainability of the test subject can be determined according to whether the genotype of the rs717742 polymorphism is “A/A” or not.

### (3) Nicotine Acetylcholine Receptor α4 Subunit (CHRNA4) Gene

For example, as detailed below, the tendency in the smoking behavior of a test subject can be determined by directly or indirectly examining rslO44397 polymorphism that exists in the fifth exon of the CHRNA4 gene.

More specifically, when the test subject (genotype A/A or G/A: an A allele carrier) had A alleles, in which the base on the polymorphism site is “A”, the following tendency with respect smoking behavior were indicated.

(i) Nicotine dependence: weak

In contrast, when the test subject (genotype G/G) had homozygous alleles having a “G” base on the polymorphism site (G allele), the following tendency with regard to smoking behavior was obtained.

(i) Nicotine dependence: weak

Based on these examination results, the tendency in the smoking behavior of the test subject can be determined according to whether the genotype of the rs1044397 polymorphism is “G/G”, or an A allele carrier (A/A or G/A).

Further, as detailed below, the tendency in the smoking behavior of a test subject can be determined by directly or indirectly examining the rs2273504 polymorphism that exists in the second intron of the CHRNA4 gene.

More specifically, when the test subject (genotype G/G or G/A: a G allele carrier) had alleles (G allele) having a “G” base on the polymorphism site, the following test result was obtained.

(i) Nicotine dependence: strong

When the test subject (genotype A/A) had homozygous A alleles, in which the base on the polymorphism site is “A”, the following test result was obtained.

(i) Nicotine dependence: strong

Based on these examination results, the tendency in the smoking behavior of the test subject can be determined according to whether the genotype of the rs2273504 polymorphism is A/A, or a G allele carrier (G/G or G/A).

The present invention is a process of determining a tendency in the smoking behavior of a test subject based on the results of an examination for the above-mentioned single or plural gene polymorphisms; however, the present invention is not limited to direct examination of each polymorphism gene. For example, the tendency in the smoking behavior of a test subject can also be determined using indirect examination of the gene polymorphisms, i.e., examination of other gene polymorphisms that are genetically linked to the target gene polymorphism and form a haplotype. Moreover, as the correlation between the gene polymorphisms related to the genes (1) to (3) and individual differences in smoking behavior has been proved, the tendency in the smoking behavior of the test subject can also be determined by examining the other gene polymorphisms in the genes (1) to (3).

Regarding the examination of the other gene polymorphisms in the genes (1) to (3), the gene polymorphism to be examined is not limited to a single nucleotide polymorphism (SNP). For example, the target polymorphism may be a polymorphism derived from the presence or absence of a defective portion; more specifically, an insertion type polymorphism having a certain base sequence of two or more bases, or a deletion type polymorphism which does not have said base sequence. As such, the determination method of the present invention may be carried out with respect to a single nucleotide polymorphism or a polymorphism derived from the presence or absence of a defective portion, or still another kind of gene polymorphism such as transposition-type polymorphism.

For example, the examination and determination of gene polymorphism is performed as follows. As a template, genomic DNA is prepared from a DNA sample extracted from the patient's blood, and the target gene region including the polymorphism site is amplified using PCR etc. The genotype is determined by subjecting this amplified fragment to primer extension or the like. The primer sequence used for PCR can be designed in reference to the sequences shown in Table 4 (and the sequence table) below. The genome sequence and the cDNA sequence of the aforementioned genes (1) to (3) are registered in published gene sequence databases, such as GenBank; therefore, the primer sequence can also be designed in reference to the base sequences in such published databases.

Regarding the present invention, it may be preferable that the tendency in the smoking behavior of a test subject be determined based on the examination results of plural gene polymorphisms related to the aforementioned genes (1) to (3), or in reference to a combination of the test result of gene polymorphisms related to the aforementioned genes (1) to (3) and the test results of the other polymorphisms.

It is further preferable to perform such determination using a program. The present invention includes a smoking cessation assistance program to carry out the determination. More specifically, the program of the present invention causes a computer to execute one of the following processes (i) to (v).

(i) a process of determining “nicotine dependence level”, “severity of nicotine withdrawal symptoms during smoking cessation” and/or “difficulty in sustaining smoking cessation” of a test subject, according to whether the genotype of the rs12072348 polymorphism is A/A or a C allele carrier (C/C or A/C)

(ii) a process of determining the “nicotine dependence level” of a test subject, according to whether or not the genotype of the test subject regarding LPR polymorphism is an L allele carrier group and the genotype of VNTR polymorphism is “l/s”.

(iii) a process of determining the “nicotine dependence level” of a test subject, according to whether the genotype of the rs717742 polymorphism is A/A or a T allele carrier (T/T or A/T).

(iv) a process of determining the “nicotine dependence level” of a test subject, according to whether the genotype of the rslO44397 polymorphism is G/G or a A allele carrier (A/A or G/A).

(v) a process of determining the “nicotine dependence level” of a test subject, according to whether the genotype of the rs2273504 polymorphism is A/A or a G allele carrier (G/G or G/A).

With these processes, a tendency in the smoking behavior of a test subject is determined by a computer, based on the results of gene polymorphism examination. The program of the present invention may be provided as a computer-readable recording medium storing the program. Examples of the recording media include, but are not limited to, flexible disks; hard disks; magnetic storage media such as magnetic tapes; optical storage media such as CD-ROM, CD-R, CD-RW, DVD-ROM, DVD-RAM or DVD-RW; electric storage media such as RAM or ROM; and magnetic/optical storage media such as MO.

An embodiment of the program of the present invention causes a computer provided with an input device, a storage device, an arithmetic processing unit and an output unit, to execute the following steps (a) to (b).
Step (a): in response to the input of information regarding at least one polymorphism among the polymorphisms mentioned in (i) to (v) of a test subject from the input device, determining the tendency in the smoking behavior of a test subject by the arithmetic processing unit based on the data showing the correlation between the polymorphism and the tendency in smoking stored in the storage device.
Step (b): displaying the determination result obtained in Step (a) by the output device.

In the present invention, the computer preferably combines two or more of the processes (i) to (v) to determine a tendency in the smoking behavior of a test subject so as to increase the accuracy of the determination.

[2]

In the present invention, the method of examining the gene polymorphisms of the aforementioned genes (1) to (3) is not particularly limited. For example, any known method that enables direct or indirect examination of a polymorphism in the gene, or any recently developed method can be used. The aforementioned method of examining the gene polymorphism by way of PCR is well known for its simplicity and accuracy. This method is briefly explained below.

The DNA sample used for the examination can be obtained through purification and extraction of DNA taken from arbitrary organs, tissues, and cells (including cells in blood or amniotic fluid, and cells prepared by cultivating the extracted organs, tissues etc.) of a test subject according to a general process. The DNA purification/extraction step may be simplified or omitted insofar as the gene amplification by PCR is possible.

Next, the gene region including the polymorphism site is amplified by performing PCR using the genomic DNA in the DNA sample prepared as a template, thereby identifying the genotype based on the polymorphism site (gene typing). Then, the genotype of the amplified fragment is measured by way of primer extension, PCR-RFLP, length measurement using electrophoresis, etc.

The conditions of the above-mentioned PCR and the reagent/primer used in PCR are not particularly limited. The examination of gene polymorphism is also not limited to PCR. For example, any known or published methods of directly or indirectly examining polymorphisms in genes, including the method of verifying the base of a single nucleotide polymorphism (SNP) (SNP typing), and a method of verifying a polymorphism derived from the presence or absence of a defective portion. (refer to Post-Sequence no Genome Kagaku (Post-Sequencing Genome Science), SNP Idensitakei no Senryaku (Strategy of SNP Gene Polymorphism), Nakayama Shoten Co. Ltd.)

As another example, a method using a gene polymorphism examination tool, such as a DNA chip can be utilized. In this method, a DNA chip (or a similar tool) is first prepared as follows. A probe for verifying the gene polymorphisms related to the aforementioned genes (1) to (3) and optionally a probe for verifying other gene polymorphisms is placed on a DNA chip. Using such a DNA chip (or a similar tool), the target polymorphism is detected by detecting a signal of hybridization between the probe and genetic material from a test subject. An oligonucleotide having the base of the polymorphism site and some neighboring base sequences or the complementary sequence thereof may be used as the probe. The “DNA chip” in this method generally indicates a synthetic DNA chip that contains a synthetic oligonucleotide as a probe; however, a sticker-type DNA microarray that uses a cDNA (PCR product etc.) as a probe can also be used. Such a DNA chip containing a probe for gene polymorphism verification for the aforementioned genes (1) to (3) can serve as a smoking cessation assistance chip.

Other examples of gene polymorphism examination methods include a point mutation detecting method such as PCR-SSCP, allele specific PCR, etc., and amplification methods other than PCR such as RCA. After the DNA amplification, the gene typing can be performed by directly determining the base sequence of the amplified fragment using a base sequence determining device (sequencer) or the like.

The examination of gene polymorphism may be carried out with respect to polymorphisms in the coding sequences that encode proteins, or with respect to those in the intron sequences or control sequences. If the polymorphism (mutation) is the mutation in the code sequence, it (mutation) can be detected by cDNA prepared from RNA or mRNA. If the polymorphism (mutation) includes amino acid substitution, it (mutation) can be detected based on the amino acid sequence of the protein or the like.

The gene polymorphism examination kit of the present invention is a kit for directly or indirectly examining gene polymorphisms related to the aforementioned genes (1) to (3), and the kit includes a primer or a probe designed based on the sequences of these genes or neighboring sequences. The kit may include one or two more items selected from: (i) an enzyme or reagent used for the preparation of a sample; (ii) an enzyme or reagent used for the reverse transcription; (iii) an enzyme or reagent used for PCR; and (iv) an enzyme or reagent used for the determination of base sequences.

### Example

The following describes an Example of the present invention. However, the present invention is not limited to the Example below.

### 1. Examination Method

### (1) Test Subject

The test subjects were 120 adult male outpatients of Osaka Regional Taxation Bureau clinic and Funamoto clinic; some are smokers (some are outpatients of a smoking cessation program) and the remaining test subjects have a smoking history.

### (2) Evaluation of Smoking Behavior

The test subjects were assessed for their smoking habits, based on the “number of cigarettes per day” and “the time to the first smoking after wake-up”, as shown in Table 1 below. Based on the values of these items, an HSI (Heaviness of Smoking Index) score was calculated for each test subject as an index of “nicotine dependence”. An HSI score of 5 or more was determined to be “nicotine dependence=strong”. The HSI is an index used as a simple evaluation of nicotine dependence. In this embodiment, HSI was computed according to the method disclosed in “Addict Behav 2004; 29:1207-12”. The evaluation of nicotine dependence usually refers to the gross of the “Fagerstrom Test for Nicotine Dependence (FTND)”; however, since the two items in Table 1 are the same as those of FTND, a high-relativity to the gross of the FTND (Drug Alcohol Depend 2003; 71:1-6) is ensured.

TABLE 1
HSI (Heaviness of Smoking Index) (Heatherton, 1989)

| Questions | Answers (scores) |
|---|---|
| How long is it before you smoke | less than 5 minutes (3) |
| a cigarette after you wake up? | 6 to 30 minutes (2) |
| | 31 to 60 minute (1) |
| | more than 61 minutes (0) |
| How many cigarettes do you | more than 31 (3) |
| smoke in a day? | 21 to 30 (2) |
| | 11 to 20 (1) |
| | less than 10 (0) |

Further, for the patients of the smoking cessation program, the “nicotine withdrawal symptom during smoking cessation” was evaluated as Withdrawal Score 1 (severe, weak). Moreover, as Withdrawal Score 2, the nicotine withdrawal symptom was classified into four levels according to “nicotine withdrawal symptom” in the category of “inappropriate use of nicotine” in the “Diagnostic and Statistical Manual of Mental Disorders (DSMIV)” provided by American Psychiatric Association, (Questionnair by DSMIV) to evaluate the severity of “nicotine withdrawal symptom” (Table 2) of each patient. Failure/success of smoking cessation was determined according to whether the test subject carried through two consecutive month smoking cessation or not.

TABLE 2
Withdrawal score 2 (questionnaire by DSMIV)
Comprehensive Evaluation (0, 1, 2, 3, 4)*

| Symptoms | Degree* | | | | |
|---|---|---|---|---|---|
| craving for nicotine (smoking) | 0 | 1 | 2 | 3 | 4 |
| uneasiness | 0 | 1 | 2 | 3 | 4 |
| quick temper, frustration, anger | 0 | 1 | 2 | 3 | 4 |
| difficulty in concentration | 0 | 1 | 2 | 3 | 4 |
| insomnia | 0 | 1 | 2 | 3 | 4 |
| discomfort, dysphoria | 0 | 1 | 2 | 3 | 4 |
| increase in appetite or weight | 0 | 1 | 2 | 3 | 4 |
| cold sweat | 0 | 1 | 2 | 3 | 4 |
| decrease in cardiac rate | 0 | 1 | 2 | 3 | 4 |

*1. Not at all 2. Very little 3. Moderate 4. Strong 5. Very Strong

(3) Selection of Candidate Gene Polymorphisms

18 genes and 37 gene polymorphisms were selected, using the following factors, as candidate gene polymorphisms, as shown in Table 3.
(i) Possibility of influence on nicotine dependence due to central nicotine action.
(ii) Possibility or actual reports of a certain influence on the molecular mechanism, such as amino acid mutation.
(iii) About 10% or higher frequency in Japanese.
(iv) Actual reports of relevance to smoking, such as smoking behavior or nicotine withdrawal symptoms, in the smoking-related study.

TABLE 3

| | | Number of |
|---|---|---|
| | | polymorphisms |
| Molecules | | examined |
| acetylcholine | nicotinic acetylcholine | 4 |
| | receptor α4 subunit | |
| | Nicotinic acetylcholine | 4 |
| | receptor β2 subunit | |
| dopamine | dopamine receptor D1 | 1 |
| | dopamine receptor D2 | 3 |
| | dopamine receptor D3 | 1 |
| | dopamine transporter | 3 |
| serotonin | serotonin receptor 1A | 1 |
| | serotonin receptor 2A | 2 |
| | serotonin receptor 2C | 4 |
| | serotonin receptor 3A | 2 |
| | serotonin transporter | 3 |
| biosynthetic/degradation | Tyrosine hydroxylase | 1 |
| system | | |
| | Tryptophane hydroxylase 2 | 1 |
| | Monoamine oxidase B | 1 |
| Others | Cholecystokinin | 1 |
| | cholecystokinin | 1 |
| | receptor A | |
| | Cannabinoid 1 receptor | 1 |
| | GABA_{B} receptor | 3 |

### (4) Preparation of Genomic DNA

5 ml of peripheral venous blood was extracted from a test subject using an EDTA blood collecting tube, and genomic DNA was prepared using a QIA amp DNA Blood Maxi kit (product name, QIAGEN), according to the manufacture's protocol.

### (5) Determination of Gene Polymorphism

The genomic DNA of the test subject was examined for the existence of polymorphisms in the candidate genes of Table 3.

The PCR product obtained by using the genomic DNA as a template was subjected to digestion by restriction endonuclease by adjusting the number of restriction endonuclease units and conditions, followed by electrophoresis using an agarose gel of an appropriate concentration according to the length of the resulting fragment, thereby confirming the gene polymorphism. The procedures of genotyping, primer sequencing, restriction endonuclease, and conditions in PCR, etc. used for this confirmation process are shown in the following and in Table 4, only for a CYP2A6 gene polymorphism and for the gene polymorphism which showed in the test result (described later) correlation with the individual differences in smoking behavior.

### (i) Determination of CYP2A6*4

Determination of CYP2A6*4 was performed by way of PCR-RFLP according to the method provided by M. Nakajima et al. (FEBS Lett 2004; 569:75-81.). The PCR was performed using 45 ng of genomic DNA, 0.4 μM of each primer, GeneAmp™ l× PCR Buffer (10 mM Tris/HCl (pH8.3), MgCl₂, 1.5 mM), 0.24 mM of each dNTP, and 1U of AmpliTaq™ DNA Polymerase (Applied Biosystems). The gross amount was 25 μl.

### (ii) Determination of CYP2A6*9

Determination of CYP2A6*9 was performed by way of allele-specific PCR according to the method provided by Yoshida, R et al. (Clin Pharmacol Ther 2003; 74:69-76). PCR was performed using 45 ng of genomic DNA, 0.4 μM of each primer, GeneAmp™ 1× PCR Buffer II, 2 mM of MgCl, 0.2 mM of each dNTP, and 1.25U of AmpliTaq Gold™ DNA Polymerase (Applied Biosystems). The gross amount was 25 μl. During PCR, the presence/absence of fragment amplification was detected, as an index of the determination, using an ABI PRISM 7700 sequence detector (Applied Biosystems) and SYBR Green I (Molecular Probes) as a fluorescent marker.

(iii) Determination of 5HTTLPR

Determination of 5HTTLPR was performed using PCR-RFLP according to the method of by Heils, A et al. (J Neurochem 1996; 66:2621-4). PCR was performed using 30 ng of genomic DNA, 5% dimethyl sulfoxide, 0.4 μM of each primer, GeneAmp™ 1× PCR Buffer, dNTPs;dGTP/7-deaza-2′-dGTP=1:1, and 1U of AmpliTaq™ DNA Polymerase. The gross amount was 25 μl.

### (iv) Determination of 5HTTVNTR

Determination of 5HTTVNTR was performed by way of PCR-RFLP according to the method of Ogilvie, A et al. (Lancet 1996; 347:731-3). PCR was performed using 30 ng of genomic DNA, 0.4 μM of each primer, GeneAmp™ 1× PCR Buffer, 0.2 mM of each dNTP, 1U of DNA Polymerase. The gross amount was 25 μl.

(v) Determination of 5HTT rs717742

Determination of 5HTT rs717742 was performed by way of PCR-RFLP. The PCR was performed using 45 ng of genomic DNA, 0.4 μM of each primer, GeneAmp™ 1× PCR Buffer, 0.2 mM of each dNTP, 1U of AmpliTaq Gold™ DNA Polymerase. The gross amount was 25 μl.

### (vi) Determination of CHRNB2A10160C

Determination of CHRNB2A10160C was performed by way of PCR-RFLP. PCR was performed using 60 ng of genomic DNA, 0.48 μM of each primer, GeneAmp™ 1× PCR Buffer, 0.2 mM of each dNTP, and 1U of AmpliTaq Gold™ DNA Polymerase. The gross amount was 25 μl. Another determination was performed using primer extension.

(vii) Determination of CHRNA4 rslO44397

Determination of CHRNA4 rslO44397 was performed by way of the TaqMan method. PCR was performed using 30 ng of genomic DNA, 1×TaqMan™ universal PCR Master Mix (Applied Biosystems), 40×Assay Mix (Applied Biosystems). The gross amount was 20 μl. Another determination was performed using primer extension method.

(viii) Determination of CHRNA4 rs2273504

Determination of CHRNA4 rs2273504 was performed by way of the TaqMan method. PCR was performed using 30 ng of genomic DNA, 1× TaqMan™ universal PCR Master Mix (Applied Biosystems), 40× Assay Mix (Applied Biosystems). The gross amount was 20 μl. Another determination was performed using primer extension method.

TABLE 4

| | | | | | | | PCR condition |
|---|---|---|---|---|---|---|---|
| | | | restriction | | | | Denature/Anealing/ |
| gene | polymorphism | genotyping | enzyme | primer | Sequence | (SEQ. No.) | Extension/cycle |
| CYP2A6*4 | deletion | PCR-RFLP | Eco8lI | F | 5′-TTTGTGTCAGGAGAATCAAAC-3′ | (1) | 94° C. 30 sec/53° C. 30 sec/ |
| | | | StuI | R | 5′-TGTAAAATGGGCATGAACGCCC-3′ | (2) | 72° C. 2 min/30 cycle |
| CYP2A6*9 | -48T/G | Allele-specific PCR | | F | 5′-GATTCCTCTCCCCTGGAAC-3′ | (3) | 94° C. 30 sec/66° C. 30 sec/ |
| | | | | R-wild | 5′-GGCTCGGGTGGTTTGCCTTTA-3′ | (4) | 72° C. 25 sec/35 cycle |
| | | | | R-mutant | 5′-GGCTGGGGTGGTTTGCCTTTCC-3′ | (5) | |
| CHRNA4 | rs1044397 | TaqMan | | | Assay ID: C_25765467_10* | | |
| | Ala553Ala | Primer | | 1st-PCR Primer | 5′-TCTGCAATGTACTGGACGCC-3′ | (6) | |
| | | extension | | 2nd-PCR Primer | 5′-ACGGTCAAGACCCGCAGCAC-3′ | (7) | |
| | rs2273504 | TaqMan | | | Assay ID: C_16178933_10* | | |
| | | Primer | | 1st-PCR Primer | 5′-AGCAAGGCAGGTGGGCAGAA-3′ | (8) | |
| | | extension | | 2nd-PCR-Primer | 5′-AGGAAACTGGAGAGGAGTCG-3′ | (9) | |
| CHRNB2 | rs12072348 | PCR-RFLP | DdeI | F | 5′-GGTATATAACAGGCACAGTTGCTA-3′ | (10) | 94° C. 30 sec/58° C. 30 sec/ |
| | | | | R | 5′-GGAGACAGCCACTGAGCATC-3′ | (11) | 72° C. 30 sec/38 cycle |
| | A10160C | Primer | | 1st-PCR Primer | 5′-TTTGCATCCCCAGTGTGTAG-3′ | (12) | |
| | | extension | | 2nd-PCR Primer | 5′-AGTCAGCAGCTGAAGAACTC-3′ | (13) | |
| SLC6A4 | LPR | PCR | | F | 5′-GGCGTTGCCGCTCTGAATTGC-3′ | (14) | 95° C. 30 sec/61° C. 30 sec/ |
| (5HTT) | (20-23 hp) | | | R | 5′-GAGGGACTGAGCTGGACAACCCAC-3′ | (15) | 72° C. 1 min/35 cycle |
| | VNTR | PCR | | F | 5′-TGTTCCTAGTCTTACGCCAGTG-3′ | (16) | 94° C. 30 sec/60° C. 30 sec/ |
| | (12 bp) | | | R | 5′-GTCAGTATCACAGGCTGCGAG-3′ | (17) | 72° C. 30 sec/35 cycle |
| | rs717742 | PCR-RFLP | DdeI | F | 5′-AGTACAGACAGGGTTTCGC-3′ | (18) | 94° C. 30 sec/56° C. 30 sec/ |
| | | | | R | 5′-CCATGCCCTTCGGTTTTCTC-3′ | (19) | 72° C. 30 sec/35 cycle |

*The determination was performed according to the protocol of TapMan ™ SNP genotyping Assays (Applied Biosystems)

### (6) Statistical Analysis

The results of gene polymorphism determination and other examinations were statistically analyzed. When the result was P<0.05, the difference was regarded significant. To eliminate the influence of individual pharmacokinetic difference, test subjects with low CYP2A6 activity were excluded; as a result, 104 test subjects with high CYP2A6 activities were selected (*1/*1, *1/*9, *1/*4, *9/*9) for the analysis. The smoking behavior items, “number of cigarettes per day” and “age of smoking onset” were determined according to “Wilcoxon's signed rank sum test”. The “time to the first smoking after wake-up” and “nicotine dependence: HSI” were determined according to Chi-Square Test. With the test results, Odds Ratios (ORs) and the 95% confidence interval (95% CI) were calculated only for the gene polymorphisms of test results that showed significant differences. Further, ORs and the 95% CI were measured only for the polymorphisms that showed significant differences in the Chi-Square Test of “nicotine dependence: HSI” and each gene polymorphism, using Nonlinear Regression Analysis, which takes the test subject's age into account. In the test subject group under smoking cessation therapy, the Chi-Square Test was performed to measure “nicotine withdrawal symptom score” and “failure/success of two months' smoking cessation”; and ORs and 95% CI were found only for the items that showed significant differences. SPSS for Windows Release 11.01J, Standard Version (product name) was used for the statistical analysis.

### 2. Results

### (1) Data of Test Subjects

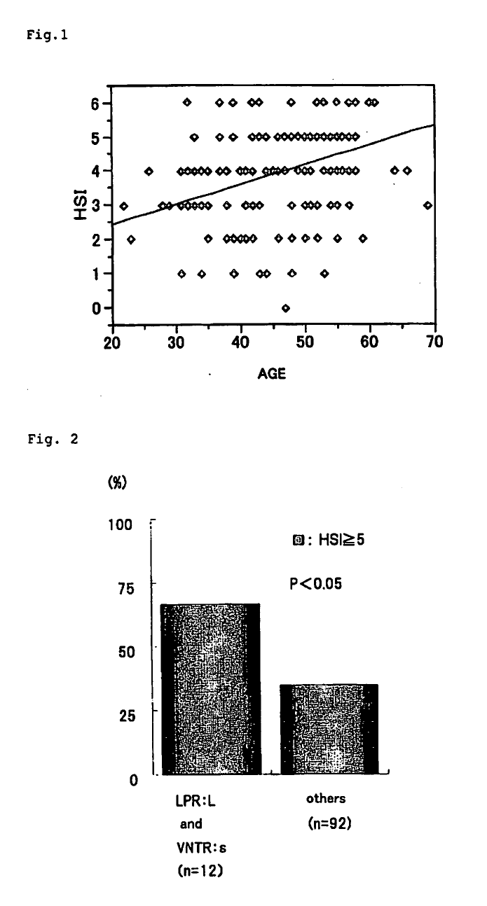

Table 5 below shows data of 120 test subjects of the present Example. As shown in Table 5, as a result of research into the factors affecting the nicotine dependence HSI, a correlation (Spearman's p=0.41 P≦0.001) was found between the nicotine dependence HSI score and the age of the test subject; HSI increases as the age increases. FIG. 1 showed a correlation between the dependence HSI score and age. Although no correlation was found between the dependence HSI score and “age of smoking onset” of the test subjects, there was a correlation (Spearman's ρ=0.041P=0.688) between the dependence HSI score and “smoking duration”; the HSI score increases as the “smoking duration” increases (Spearman's ρ=0.41P≦0.001).

TABLE 5

| | |
|---|---|
| Male | n = 120 |
| CYP2A6 High activity | n = 104 |
| CYP2A6 Low activity | n = 16 |
| Mean (SD) Age (years) | 46.48 ± 8.92 |
| Mean (SD) Age of smoking onset | 18.79 ± 1.94 |
| (years) | |
| Mean (SD) cigarettes/day | 31.35 ± 14.14 |
| Mean (SD) dependency score | 3.88 ± 1.40 |
| (HSI) | |
| Participants in smoking | n = 79 |
| cessation programm | |
| Nonsmoking at 2 months | n = 52 |
| Smoking once again | n = 27 |
| Mean (SD) withdrawal score 1 | 2.36 ± 0.65 |
| (severe, weak) | |
| Mean (SD) withdrawal score 2 | 9.39 ± 5.57 |
| (questionnaire by DSMIV) | |

### (2) Gene Polymorphism Affecting Nicotine Dependence HSI

Gene polymorphisms of 104 test subjects having high CYP2A6 activity were divided into two groups, and the frequenct difference with regard to the “severity of nicotine dependence HSI” of the two groups was analyzed using a Chi-Square Test. As a result, rslO44397 and the rs2273504 of CHRNA4 (nicotine acetylcholine receptor α4 subunit gene), A10160C polymorphism of CHRNB2 (nicotine acetylcholine receptor β2 subunit gene), and LPR polymorphism of 5HTT (serotonin transporter gene) showed significant differences (P<0.05).

Then, Logistic Regression Analysis was performed for those polymorphisms that showed significant differences (P<0.05) in the Chi-Square Test. Though a weak linearity was found between the age of the test subject and the nicotine dependence HSI, considering the fact that smoking frequency generally reaches a peak in the forties or fifties, the age was nonlinearly corrected. A change in likelihood by an increase/decrease in a single factor was multiplied with x² value of 1 degrees-of-freedom, and the model was finally chosen according to whether the significant difference (P<0.05) was found in the calculation result. Accordingly, LPR/VNTR (L/s) polymorphism of 5HTT and A10160C polymorphism of CHRNB2 were determined to be significantly involved in the “severity of nicotine dependence HSI”. Table 6 below shows P value, ORs, and 95% CI resulting from Logistic Regression Analysis of those polymorphisms. Table 7 shows some data of polymorphisms determined to be involved in nicotine dependence.

TABLE 6

| | | | | dependency score (HSI) | | | |
|---|---|---|---|---|---|---|---|
| | | aa vs aA + AA | | logistic regression analysis | | | |
| | poly- | n = 104 | | P | | | |
| gene | morphism | a | A | value | ORs | 95% CI | |
| CHRNB2 | A10160C | A | C | 0.041 | 3.39 | 1.051 | 10.941 |
| SLC6A4 | LPR/VNTR | L/s | others | 0.049 | 4.448 | 1.007 | 19.647 |
| (5HTT) | | | | | | | |

TABLE 7

| | | | SNP | |
|---|---|---|---|---|
| gene | position | polymorphism | (rs number) | effect |
| CHRNA4 | exon5 | G→A | rs1044397 | Function not known |
| | | Ala553Ala | | |
| | intron2 | G→A | rs2273504 | Function not known |
| CHRNB2 | intron5 | A10160C | rs12072348 | Function not known |
| SLC6A4 | 5′- | LPR | | Transcription |
| (5HTT) | region | (20-23 bp) | | activity |
| | | | | S/S · L/S < L/L |
| | intron2 | VNTR | | Transportation |
| | | (12 bp) | | activity |
| | | | | s > 1 |
| | intron8 | A→T | rs717742 | Function not known |

### (3) LPR/VNTR Polymorphism of 5HTT

The following shows the analysis results with regard to the relevance of LPR/VNTR polymorphism of 5HTT to the “severity of nicotine dependence HSI”. Table 8 shows distribution of LPR polymorphism and VNTR polymorphism of 5HTT among 104 test subjects having high CYP2A6 activities. As shown in FIG. 8, 12 people in the shaded region were test subjects having L allele carrier LPR polymorphisms and l/s type 5HTTLPR/VNTR(L/s) polymorphisms. The rest were test subjects having L allele non-carrier LPR polymorphisms or l/l type 5HTTLPR/VNTR(L/s) polymorphisms.

TABLE 8

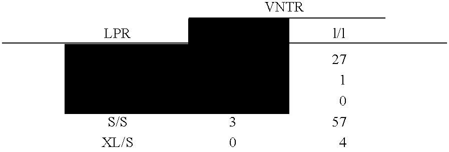

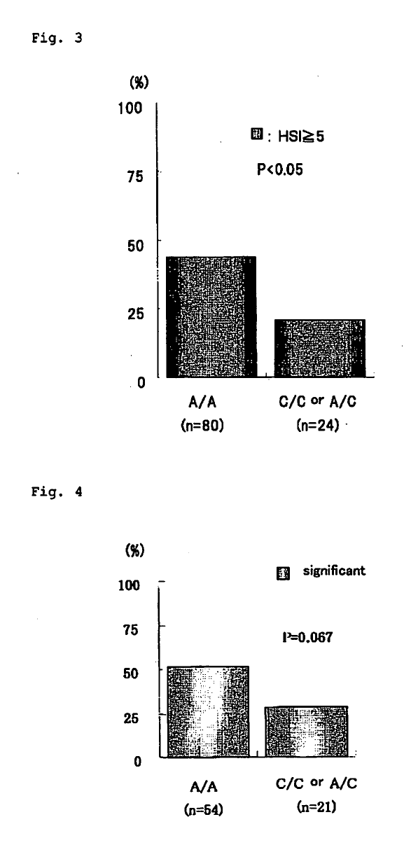

Based on Table 8, the test subjects were classified into a 5 HTTLPR/VNTR (L/s) polymorphism group (12 people), separated from the remaining test subjects (others: 92 people). For each group, the relevance to “the severity of nicotine dependence HSI” was analyzed using a Chi-Square Test and Logistic Regression Analysis. FIG. 2 shows the results. In FIGS. 2 and 3, the vertical axis indicates the ratio (%) of people whose result was HSI>=5. The following shows the results of Chi-Square Test and Logistic Regression Analysis.
Chi-Square Test: p=0.043 0Rs:3.81 95% CI: 1.06-13.64
Logistic Regression Analysis: p=0.049 0Rs: 4.449 95% CI: 1.007-19.647

As shown in the figure, the 5HTTLPR/VNTR (L/s) polymorphism group have more people showing a higher nicotine dependence (HSI≧5) than those in the other group (the remaining test subjects). This reveals that the 5HTTLPR/VNTR polymorphism is highly involved in nicotine dependence.

(4) A10160C polymorphism of CHRNB2
(4-1) nicotine dependence HSI

104 test subjects having high CYP2A6 activities are classified into two groups based on their types of A10160C polymorphisms of CHRNB2. One of the groups is A/A group consisting of 80 test subjects having homozygous A alleles, and the other is C allele carrier group consisting of 24 test subjects. For each group, the relevance with “the severity of nicotine dependence HSI” was analyzed using a Chi-Square Test and Logistic Regression Analysis. FIG. 3 shows the results. The following shows the results of the Chi-Square Test and Logistic Regression Analysis.
Chi-Square Test: p=0.043 0Rs: 2.956 95% CI: 1.004-8.70
Logistic Regression Analysis: p=0.0411 0Rs: 3.390 95% CI: 1.051-10.941

As shown in the figure, the CHRNB2 “A/A” group have more people showing a higher nicotine dependence (HSI≧5) than those in the other group (C allele carrier group). This reveals that the A10160C polymorphism of CHRNB2 is highly involved in nicotine dependence.

(4-2) Degree of Nicotine Withdrawal Symptom

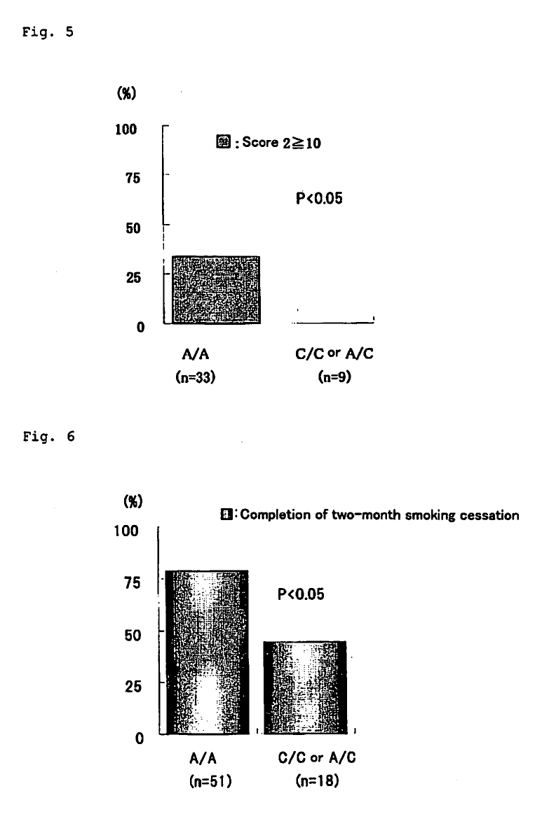

FIG. 4 shows the result of the Chi-Square Test for A10160C polymorphism of CHRNB2 and withdrawal score 1 (severe, weak) to find the relevance to “severity of nicotine withdrawal symptom”. In the figure, the vertical axis represents the ratio of those who had strong withdrawal symptoms. The CHRNB2 A/A group have more people having strong withdrawal symptoms than those in the C allele carrier groups. FIG. 5 shows the result of a similar Chi-Square Test for A10160C polymorphism of CHRNB2 and withdrawal score 2 (questionnaire by DSMIV). In FIG. 5, the vertical axis represents a ratio of the group having a high withdrawal score 2≧10. According to the result, it was found that the number of people having strong withdrawal symptoms was significantly higher in the A/A group than in the C allele-carrier group. This result matched with the result of withdrawal score 1 (severe, weak). This reveals that the A10160C polymorphism of CHRNB2 is highly involved in nicotine dependence.

### (4-3) Smoking Cessation Sustainability

FIG. 6 shows the result of another Chi-Square Test for the relevance of A10160C polymorphism of CHRNB2 to “sustainability of two months' smoking cessation”. In the figure, the vertical axis represents test subjects who succeeded in completing two months of consecutive smoking cessation. The result revealed that the rate of sustainability of two months' smoking cessation was significantly higher in the A/A group than the C allele-carrier group. This reveals that the A10160C polymorphism of CHRNB2 is highly relevant to sustainability of two months' smoking cessation. Table 9 below summarizes the results of (4-1) to (4-3).

TABLE 9

| A/A | Items | C allele |
|---|---|---|
| Strong | nicotine dependence level | weak |
| Strong | level of withdrawal symptom | weak |
| Easy | two consecutive months' | difficult |
| | smoking cessation | |

(5) The rs717742 Polymorphism of 5HTT

As shown in Table 10 below, the examination result revealed that the test subject (genotype A/T: T allele carrier) having alleles with a “T” base on the polymorphism site (T allele) showed strong nicotine dependence (i). On the other hand, the test subjects (genotype A/A) having homozygous alleles having a “A” base on the polymorphism site (A allele) showed weak nicotine dependence (i).

As such, based on these examination results, the tendency in the smoking behavior of the test subject can be determined according to whether the genotype of the rs717742 polymorphism is “A/A”, or a T allele carrier (A/A or T/A).

TABLE 10

HSI

| 5HTT rs717742 | <5 | ≧5 | SUM |
|---|---|---|---|
| A/A | 59 | 33 | 92 |
| A/T | 5 | 7 | 12 |
| SUM | 64 | 40 | 104 |

Further, as shown in FIG. 11, in the test subjects having a A/A genotype the rs717742 polymorphism, the rate of those who failed in completing two consecutive months of smoking cessation was significantly higher than the rate of those who succeeded. Accordingly, the tendency of this group with regard to “smoking cessation sustainability” was “difficult”. As such, based on these examination results, the tendency in the smoking behavior of the test subject with regard to “smoking cessation sustainability” can be determined according to whether the genotype of the rs717742 polymorphism is “A/A” or not.

TABLE 11

smoking cessation

| 5HTT rs717742 | achieved | failed | SUM |
|---|---|---|---|
| A/A | 16 | 45 | 61 |
| A/T | 5 | 3 | 8 |
| SUM | 21 | 48 | 69 |

### INDUSTRIAL APPLICABILITY

As described, the present invention relates to a process of determining a tendency in the smoking behavior of a test subject, including nicotine dependence level, nicotine withdrawal symptoms, and smoking cessation sustainability, based on the gene polymorphism. As specifically explained above, the present invention allows for the provision of important indices in individualized smoking cessation assistances/therapies; moreover, the determination result is also useful to actually plan the courses/procedures of the smoking cessation assistances/therapies.

1-10. (canceled)

11. A process of determining a tendency in the smoking behavior of a test subject, the process comprising directly or indirectly examining at least one polymorphism selected from a VNTR polymorphism in the second intron and an rs717742 polymorphism in the eighth intron of serotonin transporter (SLC6A4 (5HTT)) gene; and, based on the examination result, determining a tendency in the smoking behavior of a test subject useful for smoking cessation assistances or smoking cessation therapies.

12. The process according to claim 11, wherein the tendency in the smoking behavior of a test subject is at least one member selected from the group consisting of nicotine dependence level, severity of nicotine withdrawal symptoms, and difficulty in sustaining smoking cessation.

13. A smoking cessation assistance program for causing a computer to execute determination of a smoking behavior tendency of a test subject, according to the process of claim 11.

14. A smoking cessation assistance program for causing a computer to execute determination of a smoking behavior tendency of a test subject, according to the process of claim 12.

15. The process according to claim 11, wherein the examination of polymorphism comprises the step of amplifying a gene region including the polymorphism site using a genomic DNA prepared from the test subject as a template; and identifying a genotype based on the amplified fragment.

16. The process according to claim 12, wherein the examination of polymorphism comprises the step of amplifying a gene region including the polymorphism site using a genomic DNA prepared from the test subject as a template; and identifying a genotype based on the amplified fragment.

17. The process according to claim 11, wherein the examination of polymorphism is performed with a gene polymorphism examination tool, such as a DNA chip.

18. The process according to claim 12, wherein the examination of polymorphism is performed with a gene polymorphism examination tool, such as a DNA chip.

19. A gene polymorphism examination kit to be used in the process of claim 11.

20. A gene polymorphism examination kit to be used in the process of claim 12.


SEQUENCE LISTING (19 sequences)

SEQ ID NO: 1 - DNA, 21 bp, Artificial
  Primer
tttgtgtcaggagaatcaaac

SEQ ID NO: 2 - DNA, 22 bp, Artificial
  Primer
tgtaaaatgggcatgaacgccc

SEQ ID NO: 3 - DNA, 19 bp, Artificial
  Primer
gattcctctcccctggaac

SEQ ID NO: 4 - DNA, 21 bp, Artificial
  Primer
ggctggggtggtttgccttta

SEQ ID NO: 5 - DNA, 21 bp, Artificial
  Primer
ggctggggtggtttgcctttc

SEQ ID NO: 6 - DNA, 20 bp, Artificial
  Primer
tctgcaatgtactggacgcc

SEQ ID NO: 7 - DNA, 20 bp, Artificial
  Primer
acggtcaagacccgcagcac

SEQ ID NO: 8 - DNA, 20 bp, Artificial
  Primer
agcaaggcaggtgggcagaa

SEQ ID NO: 9 - DNA, 20 bp, Artificial
  Primer
aggaaactggagaggagtcg

SEQ ID NO: 10 - DNA, 24 bp, Artificial
  Primer
ggtatataacaggcacagttgcta

SEQ ID NO: 11 - DNA, 20 bp, Artificial
  Primer
ggagacagccactgagcatc

SEQ ID NO: 12 - DNA, 20 bp, Artificial
  Primer
tttgcatccccagtgtgtag

SEQ ID NO: 13 - DNA, 20 bp, Artificial
  Primer
agtcagcagctgaagaactc

SEQ ID NO: 14 - DNA, 21 bp, Artificial
  Primer
ggcgttgccgctctgaattgc

SEQ ID NO: 15 - DNA, 24 bp, Artificial
  Primer
gagggactgagctggacaacccac

SEQ ID NO: 16 - DNA, 22 bp, Artificial
  Primer
tgttcctagtcttacgccagtg

SEQ ID NO: 17 - DNA, 21 bp, Artificial
  Primer
gtcagtatcacaggctgcgag

SEQ ID NO: 18 - DNA, 19 bp, Artificial
  Primer
agtacagacagggtttcgc

SEQ ID NO: 19 - DNA, 20 bp, Artificial
  Primer
ccatgcccttcggttttctc